(19) United States
(12) Patent Application Publication
(10) Pub. No.: US 2010/0087623 A1
(43) Pub. Date: Apr. 8, 2010

(54) Method of extracting proteins and peptides from nano pearl powder

(76) Inventors: Feng-Di Lung, Taichung (TW); Ming Der Chen, Taichung (TW); Yung Feng Hung, Taipei (TW); Yuan Feng Feng, Taichung (TW); Yun-Chan Ting, Taipei (TW)

Correspondence Address: SAM CHEN, 7F-1, 293, ROOSEVELT ROAD, SEC 3, TAIPEI, TW

(21) Appl. No.: 12/243,977

(22) Filed: Oct. 2, 2008

(51) Int. Cl. C07K 1/34

(52) U.S. Cl. 530/344; 530/422

(57) ABSTRACT

A method of extracting proteins and peptides from nano pearl powder comprising the steps of preparing a solution containing uniformly dissolved nano pearl powder; rotating the solution to form a suspension; sifting the suspension; separating a first organic compound extract of pearl having a molecular weight more than a predetermined molecular weight and a second organic compound extract of pearl having a molecular weight less than the predetermined molecular weight from the sifted suspension respectively; and activating a first gel filter to obtain pearl proteins from the first organic compound extract of pearl, and activating a second gel filter to obtain pearl peptides from the second organic compound extract of pearl respectively.

### BACKGROUND OF THE INVENTION

1. Field of Invention

The invention relates to pearl extracts and more particularly to a method of extracting proteins and peptides from pearl powder (e.g., nano pearl powder).

2. Description of Related Art

Pearl powder used as cosmetics or food is well known. Further, pearl powder preparation methods are disclosed in prior patents. For example, U.S. Pat. No. 7,393,402 discloses a pure pearl powder preparation method. Thus, it is desirable to provide a novel method of extracting proteins and peptides from pearl powder.

### SUMMARY OF THE INVENTION

It is therefore one object of the invention to provide a method of extracting proteins and peptides from pearl powder (e.g., nano pearl powder).

The above and other objects, features and advantages of the invention will become apparent from the following detailed description taken with the accompanying drawings.

### BRIEF DESCRIPTION OF THE DRAWINGS

FIG. 1 is a flowchart depicting a process of extracting proteins and peptides from pearl powder according to the invention;

FIG. 2 is an exploded view of Centriprep Centrifugal Filter;

FIG. 3 depicts ammonium sulfate deposit according to the invention;

FIG. 4 is a side elevation of gel filter according to the invention;

FIG. 5 plots protein distribution percentage versus fraction for nano pearl powder utilized in the invention;

FIG. 6 plots protein distribution percentage versus fraction for micro pearl powder utilized in the invention;

FIG. 7 is an SDS-PAGE picture showing deposited proteins being stored in fraction 1 to fraction 6 according to the invention;

FIG. 8 is an SDS-PAGE picture showing deposited proteins being stored in fraction 7 to fraction 13 according to the invention;

FIG. 9 is an SDS-PAGE picture showing proteins extracted from micro pearl powder having a molecular weight more than 5 kDa, proteins extracted from micro pearl powder having a molecular weight less than 5 kDa, proteins extracted from nano pearl powder having a molecular weight more than 5 kDa, and proteins extracted from nano pearl powder having a molecular weight less than 5 kDa according to the invention;

FIG. 10 plots cell mobility (% of control) versus mg/mL for pearl 1 to 4 utilized in the invention;

FIG. 11 plots inhibition (%) versus time (minute) for pearl 1 to 4 and arbutin utilized in the invention;

FIG. 12 is a flowchart depicting a process of evaluating activation capabilities of the first organic compound extract of pearl and the second organic compound extract of pearl according to the invention; and

FIG. 13 is a flowchart depicting a process of further evaluating activation capabilities of the first organic compound extract of pearl and the second organic compound extract of pearl according to the invention.

### DETAILED DESCRIPTION OF THE INVENTION

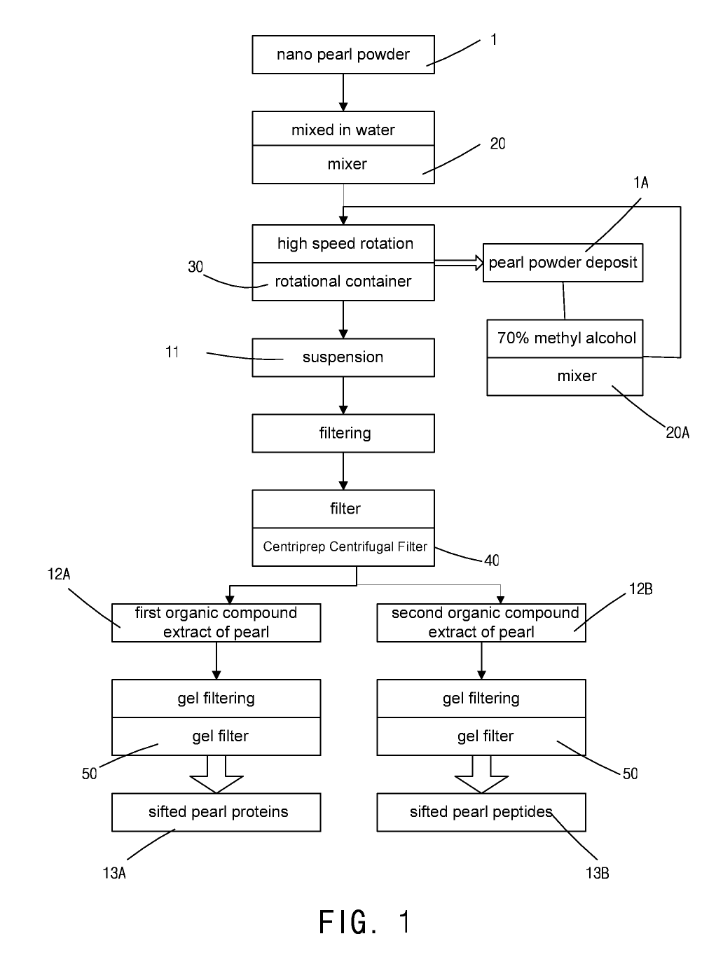

Referring to FIG. 1, a flow chart of the invention is illustrated. Nano pearl powder 1 (e.g., 20 g) is poured into water (e.g., 200 ml) to be uniformly mixed by activating a mixer 20 in room temperature for 12 hours. Next, pour the uniformly mixed solution into a rotational container 30 which is adapted to rotate in about 3,000 rpm. Hence, a suspension 11 with nano pearl powder is obtained. The suspension 11 is next sifted to separate the coarse from the fine particles. A Centriprep Centrifugal Filter 40 is employed to filter out first organic compound extract of pearl 12A having a molecular weight more than 5 kDa and second organic compound extract of pearl 12B from the sifted suspension 11 having a molecular weight less than 5 kDa respectively.

For the first organic compound extract of pearl 12A, a gel filter 50 is employed to obtain sifted pearl proteins 13A. For the second organic compound extract of pearl 12B, a gel filter 50 is employed to obtain sifted pearl peptides 13B.

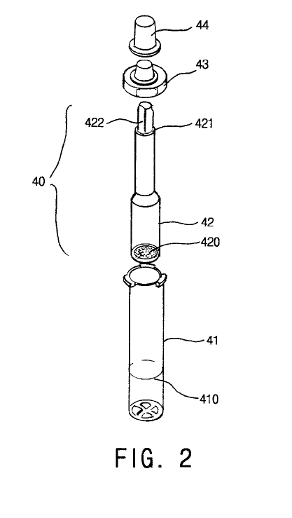

Referring to FIG. 2 in conjunction with FIG. 1, the Centriprep Centrifugal Filter 40 comprises a cylindrical shell 41 having a full marker 410 and an open top, the shell 41 being filled with the suspension 11 with the coarse being sifted out, a stepped-diameter liquid reservoir 42 adapted to fit in the shell 41 and comprising a bottom sieve 420, an upper shoulder 421, and a longitudinal discharge opening 422, a retaining ring 43 secured to the top of the shell 41 with the discharge opening 422 passing through, and a cap 44 sealingly secured to the retaining ring 43.

The Centriprep Centrifugal Filter 40 is capable of rotating in about 3,000 rpm. Hence, the suspension 11 may be sifted through the sieve 420. As a result, the first organic compound extract of pearl 12A having a molecular weight more than 3 kDa and the second organic compound extract of pearl 12B having a molecular weight less than 3 kDa can be obtained.

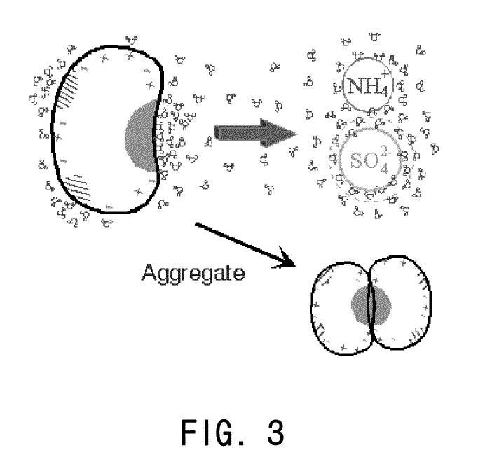

The gel filter involves the following steps. The prepared pearl solution (e.g., 10 mg/1 mL) is dropped into ammonium sulfate solution after shaking. Hence, protein is deposited. The ammonium sulfate solution is contained in a 50 mL tube which is then rotated in about 14,000 rpm at 4° C. for about 40 minutes. The deposited material is collected prior to pouring into a two-liter container full of water. Water in the container is replenished every 12 hours. Protein is filtered out of the gel after 48 hours. This process may be best described by referring to FIG. 3. Ammonium sulfate is neutral and is capable of absorbing much water. That is, the absorbed water will bind with ions of ammonium sulfate to expose non-polar zones of protein molecules. Hence, protein molecules may be combined together to deposit.

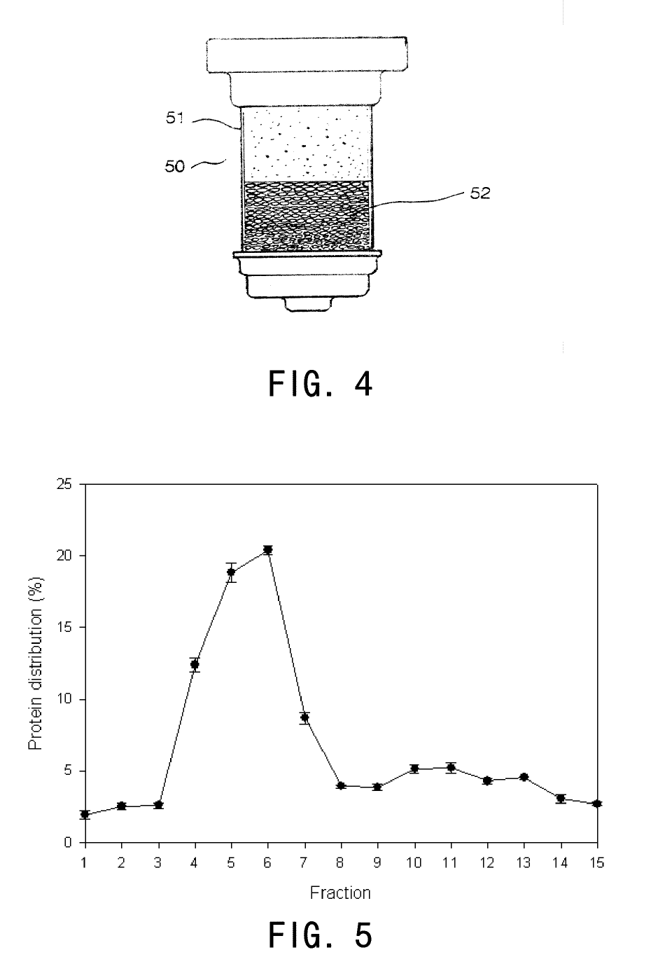

Referring to FIG. 4 in conjunction with FIG. 1, the gel filter may be done by means of a gel filter 50. The gel filter 50 comprises a cylindrical shell 51 filled with porous gel particles 52 such as polydextran gel. Coarse particles are separated from the shell 51 by quickly flowing out of the shell 51. Fine particles are contained in the shell 51 prior to slowly flowing out of the shell 51. Hence, coarse and fine particles are separated. Gel may be implemented as polydextran gel (e.g., Sephadex G25). Sephadex G25 is adapted to sift out molecules having a molecular weight less than 5 kDa. Hence, protein molecules in pearl powder can be sifted into one group having a molecular weight more than 5 kDa and the other grouped having a molecular weight less than 5 kDa.

The filtered protein is mixed with 10 mM KH₂PO₄ in a container. Next, pour 1 mL mixture into a tube called fraction. There are 15 tubes, i.e., fraction 1 to fraction 15 being obtained.

TABLE 1

| | Fraction | | | | |
|---|---|---|---|---|---|
| | 1 | 2 | 3 | 4 | 5 |
| Protein | 2 ± 0.1 | 2.5 ± 0.3 | 2.6 ± 0.2 | 12.4 ± 0.5 | 18.8 ± 0.7 |
| (%) | | | | | |
| | Fraction | | | | |
| | 6 | 7 | 8 | 9 | 10 |
| Protein | 20.4 ± 0.3 | 8.7 ± 0.4 | 3.9 ± 0.1 | 3.9 ± 0.2 | 5.1 ± 0.3 |
| (%) | | | | | |
| | Fraction | | | | |
| | 11 | 12 | 13 | 14 | 15 |
| Protein | 5.2 ± 0.4 | 4.3 ± 0.2 | 4.5 ± 0.1 | 3.1 ± 0.3 | 2.7 ± 0.2 |
| (%) | | | | | |

Referring to FIG. 5 and Table 1, protein separated from nano pearl powder is significant in fraction 4 to fraction 6 with a total protein distribution of about 51.6% and in fraction 10 to fraction 13 with a total protein distribution of about 10.3%.

TABLE 2

| | Fraction | | | | |
|---|---|---|---|---|---|
| | 1 | 2 | 3 | 4 | 5 |
| Protein | 2.6 ± 0.1 | 3.1 ± 0.2 | 3.5 ± 0.2 | 14.1 ± 0.3 | 22.9 ± 0.7 |
| (%) | | | | | |
| | Fraction | | | | |
| | 6 | 7 | 8 | 9 | 10 |
| Protein | 18.5 ± 0.6 | 5.5 ± 0.7 | 3.7 ± 0.5 | 4.2 ± 0.3 | 3.7 ± 0.4 |
| (%) | | | | | |
| | Fraction | | | | |
| | 11 | 12 | 13 | 14 | 15 |
| Protein | 3.1 ± 0.2 | 3.8 ± 0.2 | 3.2 ± 0.3 | 3.7 ± 0.4 | 4.6 ± 0.5 |
| (%) | | | | | |

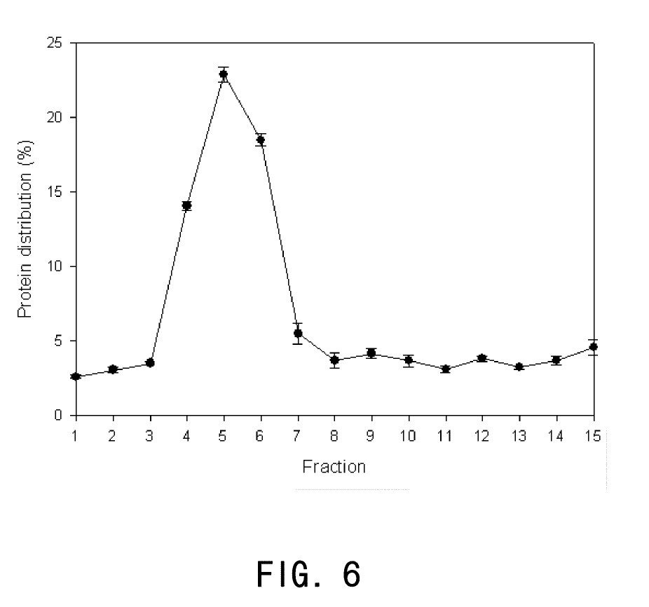

Referring to FIG. 6 and Table 2, protein separated from nano pearl powder is significant in fraction 4 to fraction 6 with a total protein distribution of about 55.5% and in fraction 15 with a protein distribution of about 4.6% but with no significant distribution from fraction 10 to fraction 14. It is found that nano pearl powder have higher protein distribution percentage than micro pearl powder in fraction 10 and fraction 11. This means that there are more protein molecules in nano pearl powder than that in micro pearl powder.

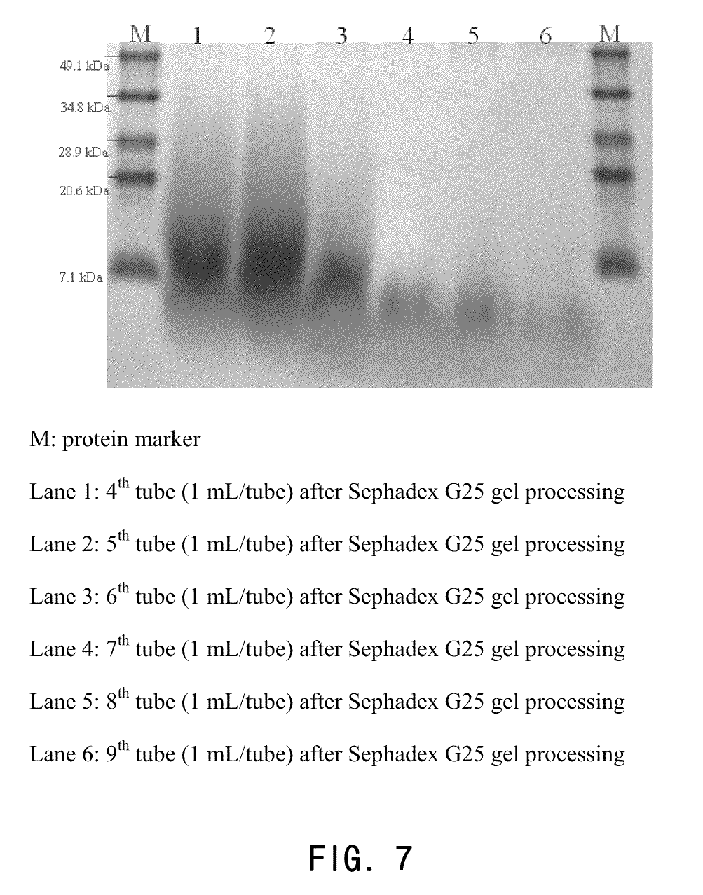

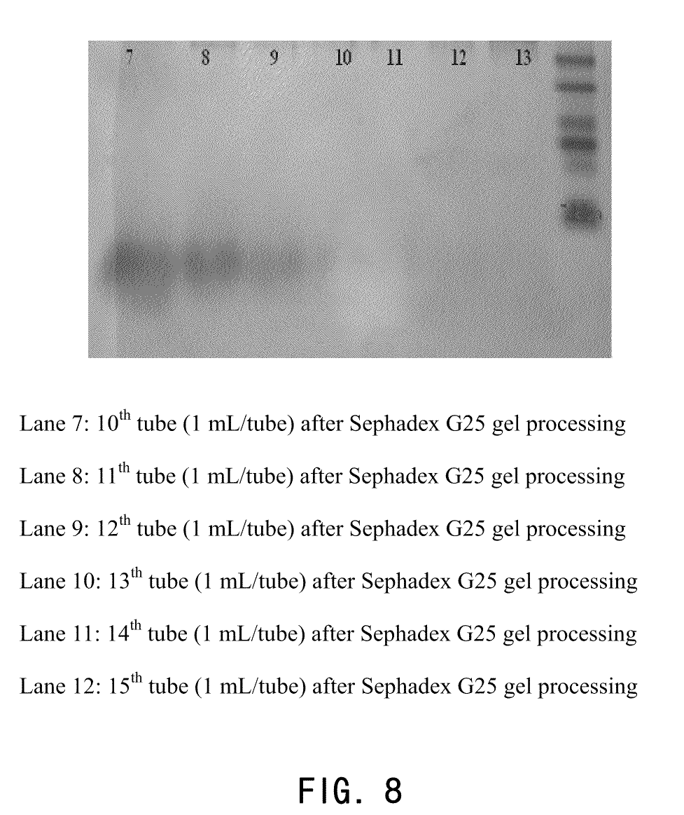

Referring to FIGS. 7 and 8, SDS-PAGE (sodium dodecyl sulfate polyacrylamide gel electrophoresis) pictures after Sephadex G25 gel processing are shown. Peptides in nano pearl powder are processed by Sephadex G25. Next, it is stored in 15 tubes each having a volume of 1 mL. The tube is called fraction. Hence, fractions 1 to 15 are obtained. Fraction 4 to fraction 6 each has a molecular weight in the range of 6 kDa to 10 kDa (collectively called fraction A). Fraction 7 to fraction 9 each has a molecular weight in the range of 4 kDa to 6 kDa (collectively called fraction B). Fraction 10 to fraction 13 each has a molecular weight in the range of 2 kDa to 3.8 kDa (collectively called fraction C).

Referring to FIG. 1 again, the pearl powder deposit 1A obtained from the rotating container 30 can be poured into a solution with 70% methyl alcohol. Next, it is mixed in a mixer 20A for 12 hours at 4° C. Next, it is poured into the rotational container 30 which is adapted to rotate in about 3,000 rpm.

The organic compounds obtained from the nano pearl powder and that obtained from the micro pearl powder according to the invention have the following characteristics.

SDS-PAGE is served for verifying the filter function. The organic compounds in micro pearl powder is slightly yellow and that in nano pearl powder is white. The organic compounds having a molecular weight less than 5 kDa is thicker than that having a molecular weight more than 5 kDa. It is thus proved that it is possible of extracting organic compounds from pearl powder according to the invention.

1 mg organic compound extract of pearl is poured into water to form a soluble organic compound extract of pearl. Next, Bradford protein binding assay is conducted to test protein. It is understood that Coomassie Brilliant Blue G-250 may bind with protein molecules of pearl extracts. Hence, Coomassie Brilliant Blue G-250 may change its color from red to blue after binding with protein molecules of pearl extracts. Thus, proteins can be easily observed if such occurs.

TABLE 3

| | Protein (μg)/organic compounds (mg) | Protein percentage (%) |
|---|---|---|
| Micro | 7.48 | 0.75 |
| Nano | 14.43 | 1.4 |

From Table 3, it is found that there is 14.43 μg of protein per 1 mg soluble organic compound extract of nano pearl. Further, the protein weight in soluble organic compound extract of micro pearl is less than that in soluble organic compound extract of nano pearl. There is 7.48 μg of protein per 1 mg soluble organic compound extract of micro pearl.

TABLE 4

| | Protein (μg)/organic compounds (mg) | Protein percentage (%) |
|---|---|---|
| Micro | 14.06 | 1.4 |
| Nano | 16.84 | 1.68 |

From Table 4, it is found that there is 16.84 μg of protein per 1 mg insoluble organic compound extract of nano pearl. Further, there is 14.06 μg of protein per 1 mg insoluble organic compound extract of micro pearl.

By comparing Table 3 with Table 4, it is found that there are more proteins in insoluble organic compound extract of either micro or nano pearl than that in soluble organic compound extract of either micro or nano pearl.

TABLE 5

| | | | |
|---|---|---|---|
| Pearl 1 | soluble organic compound extract | micro | >5 kDa |
| Pearl 2 | soluble organic compound extract | micro | <5 kDa |
| Pearl 3 | soluble organic compound extract | nano | >5 kDa |
| Pearl 4 | soluble organic compound extract | nano | <5 kDa |

Pearl powder solution is subjected to filter by employing a device called Centriprep YM3. As shown in Table 5, soluble organic compound extract of pearl is divided into two groups in which one has a molecular weight more than 5 kDa and the other has a molecular weight less than 5 kDa. In this experiment, growth rate of fiber cells in each of soluble organic compound extract of nano pearl and soluble organic compound extract of micro pearl is measured. Also, for pearl powder of the same grade growth rates of fiber cells in organic compounds having a molecular weight more than 5 kDa and in organic compounds having a molecular weight less than 5 kDa are measured.

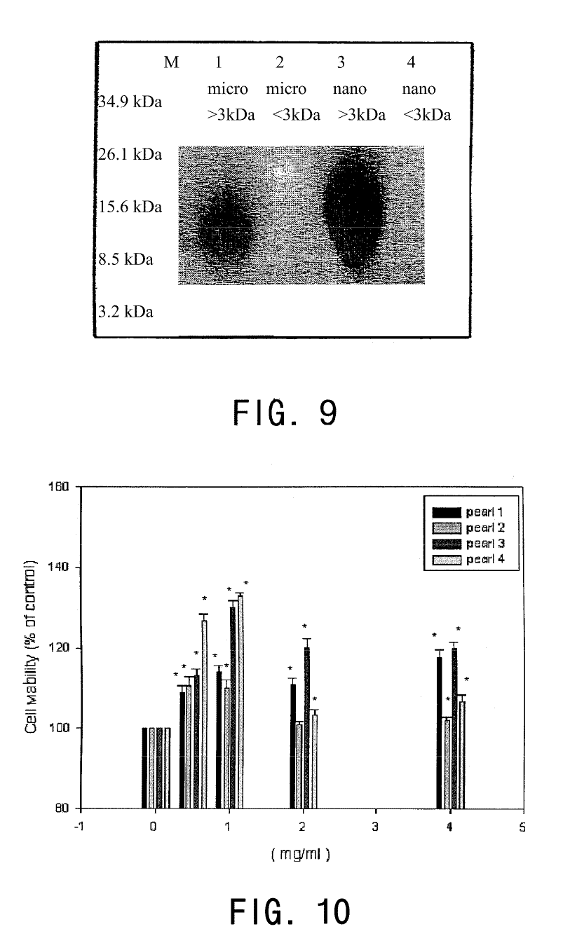

Referring to FIG. 9, it is an SDS-PAGE picture showing proteins extracted from micro pearl powder having a molecular weight more than 5 kDa, proteins extracted from micro pearl powder having a molecular weight less than 5 kDa, proteins extracted from nano pearl powder having a molecular weight more than 5 kDa, and proteins extracted from nano pearl powder having a molecular weight less than 5 kDa according to the invention.

TABLE 6

| Hs 68 | 0 mg/mL | 0.5 mg/mL | 1 mg/mL | 2 mg/mL | 4 mg/mL |
|---|---|---|---|---|---|
| Pearl 1 | 100 ± 0% | 109 ± 1.6% | 114 ± 1.6% | 111 ± 1.6% | 117.75 ± 1.7% |
| Pearl 2 | 100 ± 0% | 110.7 ± 2.1% | 110.2 ± 1.7% | 101 ± 0.8% | 102 ± 0.8% |
| Pearl 3 | 100 ± 0% | 113.2 ± 1.7% | 130.2 ± 1.7% | 120.2 ± 2.2% | 119.7 ± 1.7% |
| Pearl 4 | 100 ± 0% | 126.7 ± 1.7% | 133 ± 0.8% | 103.5 ± 1.3% | 106.7 ± 1.7% |

Referring to FIG. 10 and Table 6, for nano pearl 4 of 1 mg/mL (i.e., concentration) which is soluble organic compounds having a molecular weight of less than 5 kDa extracted from nano pearl powder, the growth rate of fiber cells is about 133% after 24 hours. For pearl 3 of 1 mg/mL which is soluble organic compounds having a molecular weight of more than 5 kDa extracted from nano pearl powder, the growth rate of fiber cells is about 130% after 24 hours.

As a comparison, for micro pearl 1 of 1 mg/mL which is soluble organic compounds having a molecular weight of more than 5 kDa extracted from micro pearl powder, the growth rate of fiber cells is about 144% after 24 hours. For micro pearl 2 of 1 mg/mL which is soluble organic compounds having a molecular weight of more than 5 kDa extracted from micro pearl powder, the growth rate of fiber cells is about 110% after 24 hours. Both growth rates are not significant.

Concentration is increased to 2 mg/mL. It is found that the growth rate is decreased to about 111% for pearl 1, decreased to about 101% for pearl 2, decreased to about 120% for pearl 3, and decreased to about 103% for pearl 4 respectively. Next, concentration is further increased to 4 mg/mL. It is found that the growth rate is increased to about 117% for pearl 1, decreased to about 102% for pearl 2, decreased to about 119% for pearl 3, and increased to about 106% for pearl 4 respectively.

It is thus concluded that growth rate of fiber cells for organic compounds is not proportional to concentration.

Pearl powder solution is subjected to filter by employing a device called Centriprep YM3. Soluble organic compound extract of pearl is divided into two groups in which one has a molecular weight more than 5 kDa and the other has a molecular weight less than 5 kDa. In this experiment, effects of tyrosinase activation (i.e., dopachrome growth) caused by each of soluble organic compound extract of nono pearl powder and soluble organic compound extract of micro pearl powder are evaluated. Also, for pearl powder of the same effects of tyrosinase activation caused by organic compounds having a molecular weight more than 5 kDa and by organic compounds having a molecular weight less than 5 kDa are evaluated.

TABLE 7

| Inhibition | | | | | |
|---|---|---|---|---|---|
| (%) | Pearl 1 | Pearl 2 | Pearl 3 | Pearl 4 | arbutin |
| 0 minute | 0 ± 0 | 0 ± 0 | 0 ± 0 | 0 ± 0 | 0 ± 0 |
| 5 minute | 9.7 ± 0.1 | 6.8 ± 0.1 | 17.6 ± 0.7 | 10.5 ± 0.8 | 36.5 ± 1.9 |
| 10 minute | 14.3 ± 1.1 | 12.3 ± 0.4 | 20.8 ± 1.3 | 13.8 ± 0.7 | 48.6 ± 0.7 |
| 15 minute | 15.2 ± 0.1 | 14.2 ± 0.4 | 23.5 ± 1.5 | 15.7 ± 0.8 | 54.3 ± 1.0 |
| 30 minute | 13.6 ± 0.5 | 14.0 ± 0 | 24.7 ± 1.0 | 14.3 ± 0.4 | 54.7 ± 0.9 |
| 45 minute | 5.7 ± 0 | 11.5 ± 0.3 | 20.9 ± 0.9 | 9.2 ± 0.6 | 37.1 ± 1.3 |
| 60 minute | 2.8 ± 0.2 | 7.72 ± 0.2 | 19.3 ± 0.5 | 4.7 ± 0.6 | 21.6 ± 1.9 |

Referring to Table 7, it shows inhibition percentage of pearl extracts with respect to tyramine acid enzyme and that of arbutin with respect to tyramine acid enzyme.

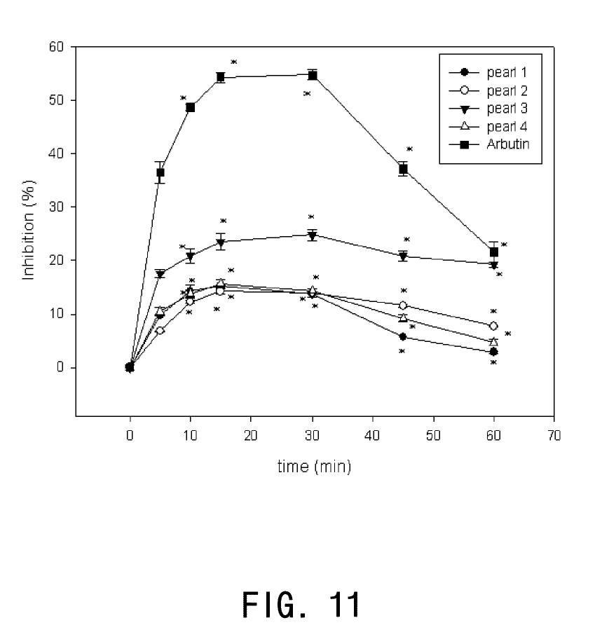

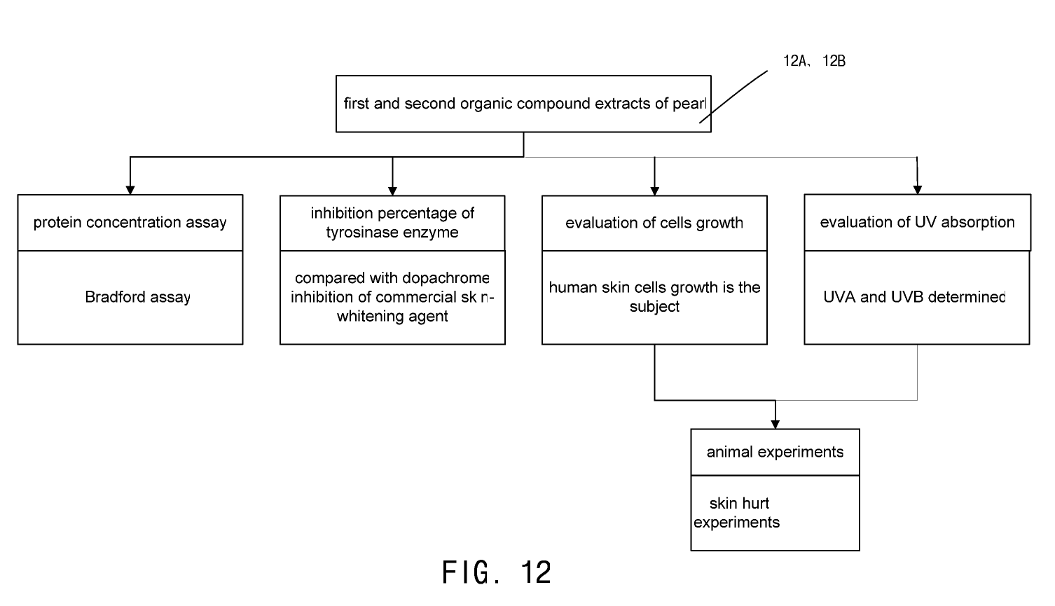

Referring to FIG. 11, it is a chart depicting inhibition percentage of pearl extracts (of 2 mg/mL) with respect to tyramine acid enzyme and that of arbutin (of 2 mg/mL) with respect to tyramine acid enzyme.

Referring to FIG. 11 and Table 7 again, experiment conditions are concentration of 1 mg/mL, temperature of 37° C., and pH value of 6.8. Next, tyramine acid of 20 mM is reacted with tyramine acid enzyme of 383 units/mL for 30 minutes. As a result, 24% inhibition of dopachrome growth is achieved. Further, 19% inhibition of dopachrome growth is achieved after one hour reaction. For the same amount of arbutin, 53% inhibition of dopachrome growth is achieved after 30 minutes of reaction. Further, 21% inhibition of dopachrome growth is achieved after one hour of reaction. Inhibition percentage of pearl 4 is about 45% of that of arbutin after 30 minutes of reaction. Further, inhibition percentage of pearl 4 is about the same as that of arbutin after one hour of reaction.

For all of pearl 1, pearl 2, pearl 3, and pearl 4, their inhibition percentage of tyramine acid enzyme is in the range of 14% to 15% after 30 minutes of reaction. Further, their inhibition percentage of tyramine acid enzyme is in the range of 2% to 7% after one hour of reaction.

Referring to FIG. 12 in conjunction with FIG. 1, each of the first organic compound extract of pearl 12A and the second organic compound extract of pearl 12B is subjected to the following four steps. The first one is protein concentration assay in which Bradford assay is conducted. There is 14.06 μg of protein per 1 mg extract. The second one is inhibition percentage of tyramine acid enzyme. Further, it is compared with the inhibition percentage of tyramine acid enzyme of one commercial type of skin-whitening agent. Hence, the growth path of melanin can be stopped, thereby significantly increasing the inhibition percentage of tyramine acid enzyme. The third one is evaluation of cells. It is found that human skin cells growth is increased significantly. The fourth one is evaluation of UV (ultraviolet) absorption in which UVA (i.e., UV absorption of the first organic compound extract of pearl 12A) and UVB (i.e., UV absorption of the second organic compound extract of pearl 12B) are determined.

Moreover, a fifth one is conducted after performing the third and fourth steps. The fifth one involves animal experiments, skin hurt experiments, etc. so as to determine its capability of skin protection.

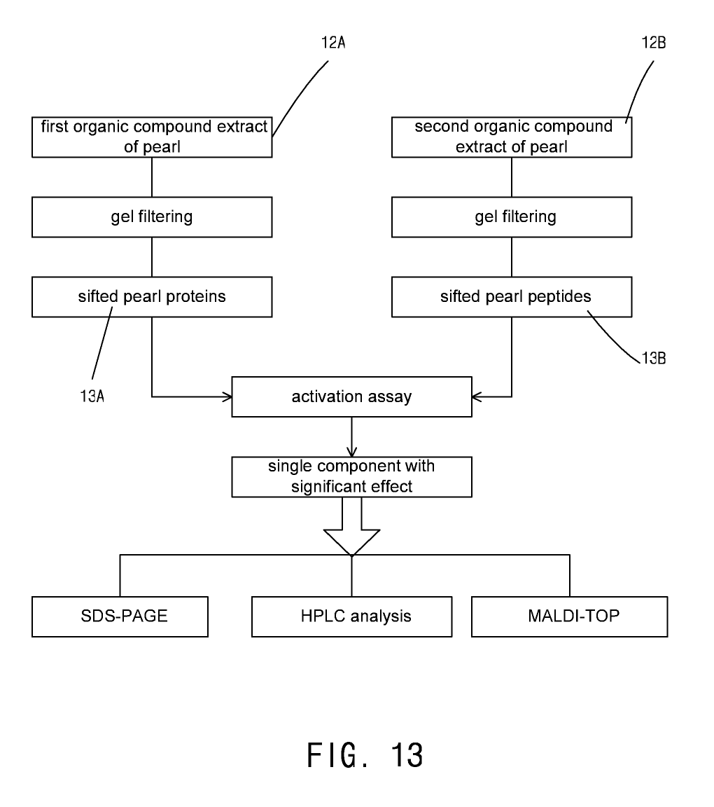

Referring to FIG. 13, for the first organic compound extract of pearl 12A, a gel filter is employed to obtain sifted pearl proteins 13A; and for the second organic compound extract of pearl 12B, a gel filter 50 is employed to obtain sifted pearl peptides 13B. Both the sifted pearl proteins 13A and the sifted pearl peptides 13B are combined to be subjected to an activation assay so as to obtain a single component with significant effect. Thereafter, SDS-PAGE, HPLC (high-performance liquid chromatography) analysis, and MALDI-TOP (matrix-assisted laser desorption/ionization-time of flight mass spectrometer) are performed respectively.

While the invention herein disclosed has been described by means of specific embodiments, numerous modifications and variations could be made thereto by those skilled in the art without departing from the scope and spirit of the invention set forth in the claims.

What is claimed is:

1. A method of extracting proteins and peptides from nano pearl powder comprising the steps of:
(a) preparing a solution containing uniformly dissolved nano pearl powder;
(b) rotating the solution to form a suspension;
(c) sifting the suspension;
(d) separating a first organic compound extract of pearl having a molecular weight more than a predetermined molecular weight and a second organic compound extract of pearl having a molecular weight less than the predetermined molecular weight from the sifted suspension respectively; and
(e) activating a first gel filter to obtain pearl proteins from the first organic compound extract of pearl, and activating a second gel filter to obtain pearl peptides from the second organic compound extract of pearl respectively.

2. The method of claim 1, wherein the predetermined molecular weight is 5 kDa.

3. The method of claim 1, wherein the rotating speed is about 3,000 rpm in step (b).

4. The method of claim 1, wherein pearl powder deposit is further obtained from step (b), and the pearl powder deposit is adapted to pour into a solution with 70% methyl alcohol and uniformly mix prior to feeding back to the solution in step (b).
